(19) United States
(12) Patent Application Publication
(10) Pub. No.: US 2024/0207204 A1
(43) Pub. Date: Jun. 27, 2024

(54) Treatment Of Type 2 Diabetes With Hepatocyte Nuclear Factor 4 Alpha (HNF4A) Agonists

(71) Applicant: Regeneron Pharmaceuticals, Inc., Tarrytown, NY (US)

(72) Inventors: Moeen Riaz, Tarrytown, NY (US); Alan Shuldiner, Tarrytown, NY (US); Joshua Backman, Tarrytown, NY (US); Jonas Bovijn, Tarrytown, NY (US); Gemma Pearson, Tarrytown, NY (US); Viktoria Gusarova, Tarrytown, NY (US)

(21) Appl. No.: 18/535,052

(22) Filed: Dec. 11, 2023

Related U.S. Application Data
(60) Provisional application No. 63/431,755, filed on Dec. 12, 2022.
(60) Provisional application No. 63/432,592, filed on Dec. 14, 2022.

(51) Int. Cl. A61K 31/137; A61K 31/165; A61K 31/713; A61P 3/08

(52) U.S. Cl. CPC A61K 31/137; A61K 31/165; A61K 31/713; A61P 3/08

(57) ABSTRACT

The present disclosure generally relates to the treatment of subjects having Type 2 diabetes and/or chronic kidney disease or at risk of developing Type 2 diabetes and/or chronic kidney disease by administering a Hepatocyte Nuclear Factor 4 Alpha (HNF4A) agonist to the subject.

### REFERENCE TO SEQUENCE LISTING

This application includes a Sequence Listing filed electronically as an XML file named 381204108SEQ, created on Dec. 6, 2023, with a size of 21,201 bytes. The Sequence Listing is incorporated herein by reference.

### FIELD

The present disclosure generally relates to the treatment of subjects having Type 2 diabetes and/or chronic kidney disease, or at risk of developing Type 2 diabetes and/or chronic kidney disease, by administering a Hepatocyte Nuclear Factor 4 Alpha (HNF4A) agonist to the subject, and to methods of identifying subjects having an increased risk of developing Type 2 diabetes and/or chronic kidney disease.

### BACKGROUND

The global epidemic of Type II diabetes is a major public health problem, as this disease is the fifth leading cause of death worldwide and a leading cause of morbidity, premature coronary heart disease, stroke, peripheral vascular disease, renal failure, and amputation. The number of individuals living with diabetes worldwide is predicted to increase from 366 million in 2011 to 552 million by 2030.

Type II diabetes is a non-insulin-dependent diabetes that is characterized by hyperglycemia due to impaired insulin secretion and insulin resistance in target tissues. Type II diabetes is typically diagnosed after the age of 40 years and is caused by the combined action of genetic susceptibility and environmental factors. Type II diabetes is associated with obesity, and it is also a polygenic disease.

In the United States, based on data from the 1999-2006 National Health and Nutrition Examination Survey (NHANES) study, an estimated 11.1 percent (22.4 million) of adults aged 20 or older have chronic kidney disease (CKD) stages 1-3. An additional 0.8 million U.S. adults aged 20 or older have CKD stage 4, and more than 0.3 million have stage 5 CKD and receive hemodialysis. Analyses of NHANES data between 1988-1994 and 1999-2004 suggest that the prevalence of CKD is rising for every CKD stage, but with a particular increase in the prevalence of individuals classified with CKD stage 3. The number of patients with stage 5 CKD requiring dialysis also has increased. It has been estimated that more than 700,000 individuals will have End Stage Renal Disease (ESRD) by 2015. Although CKD can be caused by primary kidney disease (e.g., glomerular diseases, tubulointerstitial diseases, obstruction, and polycystic kidney disease), in the vast majority of patients with CKD, the kidney damage is associated with other medical conditions such as diabetes and hypertension. In 2008, excluding those with ESRD, 48 percent of Medicare patients with CKD had diabetes, 91 percent had hypertension, and 46 percent had atherosclerotic heart disease. Other risk factors for CKD include age, obesity, family history, and ethnicity. CKD has been associated with numerous adverse health outcomes.

HNF4A is encoded by a 79 kb gene located at 20q13.12 and is present in several isoforms. HNF4A protein is 474 amino acids long and is a 53 kDa protein that functions as a nuclear receptor transcription factor which binds DNA as a homodimer. HNF4A controls the expression of several hepatic genes during the transition of endodermal cells to hepatic progenitor cells, activates the transcription of CYP2C38, represses the CLOCK-ARNTL/BMAL1 transcriptional activity, and is essential for circadian rhythm maintenance and period regulation in the liver and colon cells. HNF4A is mostly expressed in the liver, gut, kidney, and pancreatic cells and plays a role in the development of the liver, kidney, and intestines.

### SUMMARY

The present disclosure provides methods of treating a subject having Type 2 diabetes and/or chronic kidney disease, or at risk of developing Type 2 diabetes and/or chronic kidney disease, the method comprising administering an HNF4A agonist to the subject.

The present disclosure also provides methods of treating a subject having Type 2 diabetes and/or chronic kidney disease, or at risk of developing Type 2 diabetes and/or chronic kidney disease by administering a Type 2 diabetes therapeutic agent, the method comprising: determining or having determined whether the subject has an HNF4A variant nucleic acid molecule, by: obtaining or having obtained a biological sample from the subject; and performing or having performed a sequence analysis on the biological sample to determine if the subject has a genotype comprising the HNF4A variant nucleic acid molecule; and administering or continuing to administer the Type 2 diabetes and/or chronic kidney disease therapeutic agent in an amount that is the same as or less than a standard dosage amount, and/or an HNF4A agonist to a subject that is HNF4A reference or heterozygous for the HNF4A variant nucleic acid molecule; or administering or continuing to administer the Type 2 diabetes and/or chronic kidney disease therapeutic agent in a standard dosage amount to a subject that is homozygous for the HNF4A variant nucleic acid molecule; wherein the presence of the HNF4A variant nucleic acid molecule indicates the subject has a decreased risk of developing Type 2 diabetes and/or chronic kidney disease.

The present disclosure also provides methods of identifying a subject having an increased risk of developing Type 2 diabetes and/or chronic kidney disease, the method comprising: determining or having determined the presence or absence of an HNF4A variant nucleic acid molecule in a biological sample obtained from the subject; wherein: when the subject is HNF4A reference, then the subject has an increased risk of developing Type 2 diabetes and/or chronic kidney disease; and when the subject is heterozygous or homozygous for the HNF4A variant nucleic acid molecule, then the subject has a decreased risk of developing Type 2 diabetes and/or chronic kidney disease.

The present disclosure provides methods of increasing the protein level of HNF4α in a subject in need thereof, the method comprising administering an HNF4α protein encoded by an HNF4A gain-of-function variant nucleic acid molecule to the subject.

The present disclosure also provides Type 2 diabetes and/or chronic kidney disease therapeutic agents for use in the treatment or prevention of Type 2 diabetes and/or chronic kidney disease in a subject that is heterozygous or homozygous for an HNF4A variant nucleic acid molecule.

The present disclosure also provides HNF4A agonists for use in the treatment or prevention of Type 2 diabetes and/or chronic kidney disease in a subject that is HNF4A reference or heterozygous for an HNF4A variant nucleic acid molecule.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying FIGURES, which are incorporated in and constitute a part of this specification, illustrate several features of the present disclosure and are intended to exemplify non-limiting embodiments of the present disclosure.

FIG. 1 shows increased protein expression following transient transfection of mutant HNF4α in Huh7 cells; RNA expression of HNF4α in Huh7 cells 48 hours post transfection (n=4 experiments) (Panel A); representative western blot of Huh7 cells showing protein expression 48 hour post-transfection in whole cell lysate (WCL), cytosolic (Cyto) and nuclear (Nuc) fractions (Panel B); SP-1 protein expression serves as a nuclear marker, ß-actin serves as a loading control (Panel C); quantification of protein levels of HNF4α (n=3 experiments, 2 technical replicates per experiment).

### DESCRIPTION

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by ±10% and remain within the scope of the disclosed embodiments.

As used herein, the term “comprising” may be replaced with “consisting” or “consisting essentially of” in particular embodiments as desired.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the term “subject” includes any animal, including mammals. Mammals include, but are not limited to, farm animals (such as, for example, horses, cows, and pigs), companion animals (such as, for example, dogs and cats), laboratory animals (such as, for example, mice, rats, and rabbits), and non-human primates. In some embodiments, the subject is a human. In some embodiments, the human is a patient under the care of a physician.

It has been observed in accordance with the present disclosure that a rare HNF4A missense variant nucleic acid molecule (whether this variant is homozygous or heterozygous in a particular subject) associates with a decreased risk of developing Type 2 diabetes and/or chronic kidney disease. It is believed that HNF4A variant nucleic acid molecules, such as 20:44429549:C:T, have not been associated with Type 2 diabetes and/or chronic kidney disease in humans. Therefore, subjects that are HNF4A reference or heterozygous for an HNF4A variant nucleic acid molecule may be treated with an HNF4A agonist such that Type 2 diabetes and/or chronic kidney disease is inhibited or prevented, the symptoms thereof are reduced or prevented, and/or development of symptoms is repressed or prevented. It is also believed that such subjects having Type 2 diabetes and/or chronic kidney disease may further be treated with one or more Type 2 diabetes and/or chronic kidney disease therapeutic agents. In addition, the present disclosure provides methods of leveraging the presence or absence of HNF4A variant nucleic acid molecules in subjects to identify or stratify risk in such subjects of developing Type 2 diabetes and/or chronic kidney disease, or to diagnose subjects as having an increased risk of developing Type 2 diabetes and/or chronic kidney disease.

For purposes of the present disclosure, any particular subject, such as a human, can be categorized as having one of three HNF4A genotypes: i) HNF4A reference; ii) heterozygous for an HNF4A variant nucleic acid molecule; or iii) homozygous for an HNF4A variant nucleic acid molecule. A subject is HNF4A reference when the subject does not have a copy of an HNF4A variant nucleic acid molecule. A subject is heterozygous for an HNF4A variant nucleic acid molecule when the subject has a single copy of an HNF4A variant nucleic acid molecule. A subject is homozygous for an HNF4A variant nucleic acid molecule when the subject has two copies of an HNF4A variant nucleic acid molecule.

In any of the embodiments described herein, the HNF4A variant nucleic acid molecule can be any nucleic acid molecule (such as, a genomic nucleic acid molecule, an mRNA molecule, or a cDNA molecule produced from an mRNA molecule) encoding an HNF4A variant polypeptide having a partial gain-of-function, a complete gain-of-function, a predicted partial gain-of-function, or a predicted complete gain-of-function. In some embodiments, the HNF4A variant nucleic acid molecule results in increased expression or activity of HNF4A mRNA or polypeptide. In some embodiments, the HNF4A variant nucleic acid molecule is associated with an increased in vitro and in vivo response to HNF4A ligands compared with reference HNF4A. In some embodiments, the HNF4A variant nucleic acid molecule is a splice-site variant, a stop-gain variant, a start-loss variant, a stop-loss variant, a frameshift variant, or an in-frame indel variant. In some embodiments, the HNF4A variant nucleic acid molecule is a missense variant nucleic acid molecule. In some embodiments, the HNF4A variant nucleic acid molecule comprises a single nucleotide polymorphism (SNP). In some embodiments, the HNF4A variant nucleic acid molecule comprises a variation in a coding region. In some embodiments, the HNF4A variant nucleic acid molecule does not comprise a variation in a non-coding region, except for a splice acceptor region (two bases before the start of any exon except the first).

In some embodiments, the HNF4A variant nucleic acid molecule is a variant that is predicted to be beneficial to protein function (and hence, in this case, protective to the human) by in vitro prediction algorithms such as Polyphen, SIFT, or similar algorithms. In some embodiments, the HNF4A variant nucleic acid molecule is a variant that causes or is predicted to cause a nonsynonymous amino acid substitution in an HNF4A nucleic acid molecule and whose allele frequency is less than 1/100 alleles in the population from which the subject is selected. In some embodiments, the HNF4A variant nucleic acid molecule is any rare missense variant (allele frequency <0.1%; or 1 in 1,000 alleles), or any splice-site, stop-gain, start-loss, stop-loss, frameshift, or in-frame indel, or other frameshift HNF4A variant.

In some embodiments, a loss-of-function (LOF) autosomal dominant allele of HNF4A is the cause of maturity-onset diabetes of the young (MODY1) (Stoffel et al., Proc. Natl. Acad. Sci. U.S.A., 1997, 94, 13209-13214). Thus, in connection with the GOF discovery on protective effect for T2D and CKD herein, a subject having MODY1 can be treated with an HNF4A agonist.

In any of the embodiments described herein, the HNF4A variant genomic nucleic acid molecule may include one or more variations at any of the positions of chromosome 20 (i.e., positions 44,355,699-44,432,845) using the nucleotide sequence of the HNF4A reference genomic nucleic acid molecule in the GRCh38/hg38 human genome assembly (see, ENSG00000101076, ENST00000316673.9 annotated in the in the Ensembl database (URL: world wide web at “useast.ensembl.org/Homo_sapiens/Gene/Summary?g=ENSG00000101076;r=20:44355699-44432845”) as a reference sequence. The sequences provided in these transcripts for the HNF4A genomic nucleic acid molecule are only exemplary sequences. Other sequences for the HNF4A genomic nucleic acid molecule are also possible, such as in human reference genome GRCh37/hg37.

In any of the embodiments described herein, the HNF4A variant nucleic acid molecule may comprise the following genetic variation in the genomic nucleic acid molecule (referring to the chromosome:positions set forth in the GRCh38/hg38 human genome assembly): 20:44429549:C:T (rs150776703), or an mRNA molecule produced therefrom, or a cDNA molecule produced from the mRNA molecule. rs150776703 encodes the Pro437Ser HNF4A variant polypeptide.

For subjects that are genotyped or determined to be HNF4A reference, such subjects have an increased risk of developing Type 2 diabetes and/or chronic kidney disease (compared to subjects that are heterozygous or homozygous for an HNF4A variant nucleic acid molecule). For subjects that are genotyped or determined to be either HNF4A reference or heterozygous for an HNF4A variant nucleic acid molecule, such subjects can be treated with an HNF4A agonist.

In any of the embodiments described herein, the subject in whom Type 2 diabetes is prevented by administering an HNF4A agonist may be anyone at risk for developing Type 2 diabetes including, but not limited to, subjects with a genetic predisposition for developing Type 2 diabetes. Additional risk factors for Type 2 diabetes include, but are not limited to, high cholesterol, overweight, obesity, age 35 or older, race (i.e., African American, American Indian, Asian American, Hispanic/Latino, Pacific Islander, South Asian), lack of physically active, prediabetes, or have a history of gestational diabetes, or any combination thereof. In some embodiments, administering an HNF4A agonist to a subject having Type 2 diabetes may be carried out to prevent development of a more severe form of Type 2 diabetes. In any of the embodiments described herein, the methods can be used to improve Type 2 diabetes. In some embodiments, the subject is South Asian.

Symptoms of chronic kidney disease include, but are not limited to, nausea, vomiting, loss of appetite, fatigue and weakness, sleep problems, changes urination volume, decreased mental sharpness, muscle twitches and cramps, swelling of feet and ankles, persistent itching, chest pain, fluid build-up around the lining of the heart, shortness of breath, fluid build-up in the lungs, and high blood pressure (hypertension) that's difficult to control.

In any of the embodiments described herein, the HNF4A predicted gain-of-function polypeptide can be any HNF4A polypeptide having a partial gain-of-function, a complete gain-of-function, a predicted partial gain-of-function, or a predicted complete gain-of-function.

The HNF4A variant nucleic acid molecule described herein can be used with any other HNF4A variant nucleic acid molecules within any of the methods described herein to determine whether a subject has an increased or decreased risk of developing Type 2 diabetes and/or chronic kidney disease. The combinations of particular variants can form a mask used for statistical analysis of the particular correlation of HNF4A and an increased or decreased risk of developing Type 2 diabetes and/or chronic kidney disease.

In any of the embodiments described herein, the subject can have Type 2 diabetes and/or chronic kidney disease. In any of the embodiments described herein, the subject can be at risk of developing Type 2 diabetes and/or chronic kidney disease.

The present disclosure provides methods of treating a subject having Type 2 diabetes and/or chronic kidney disease or at risk of developing Type 2 diabetes and/or chronic kidney disease, the methods comprising administering an HNF4A agonist to the subject.

In some embodiments, the HNF4A agonist comprises alverine, N-trans caffeoyltyramine (NCT), or a small activating ribonucleic acid (saRNA). In some embodiments, the HNF4A agonist comprises alverine. In some embodiments, the HNF4A agonist comprises NCT. In some embodiments, the HNF4A agonist comprises an saRNA. Examples of saRNA molecules include, but are not limited to those disclosed in U.S. Patent Application Publication No. US 2022/0090074 and Huang et al., Mol. Ther. Nucl. Acids, 2020, 19, 361-370).

The saRNA molecules can comprise RNA, DNA, or both RNA and DNA. The saRNA molecules can also be linked or fused to a heterologous nucleic acid sequence, such as in a vector, or a heterologous label. For example, the saRNA molecules can be within a vector or as an exogenous donor sequence comprising the saRNA molecule and a heterologous nucleic acid sequence. The saRNA molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3×FLAG, 6×His or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

The saRNA molecules can comprise, for example, nucleotides or non-natural or modified nucleotides, such as nucleotide analogs or nucleotide substitutes. Such nucleotides include a nucleotide that contains a modified base, sugar, or phosphate group, or that incorporates a non-natural moiety in its structure. Examples of non-natural nucleotides include, but are not limited to, dideoxynucleotides, biotinylated, aminated, deaminated, alkylated, benzylated, and fluorophor-labeled nucleotides.

The saRNA molecules can also comprise one or more nucleotide analogs or substitutions. A nucleotide analog is a nucleotide which contains a modification to either the base, sugar, or phosphate moieties. Modifications to the base moiety include, but are not limited to, natural and synthetic modifications of A, C, G, and T/U, as well as different purine or pyrimidine bases such as, for example, pseudouridine, uracil-5-yl, hypoxanthin-9-yl (I), and 2-aminoadenin-9-yl. Modified bases include, but are not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (such as, for example, 5-bromo), 5-trifluoromethyl and other 5-substituted uracils and cytosines, 7-methylguanine, 7-methyladenine, 8-azaguanine, 8-azaadenine, 7-deazaguanine, 7-deazaadenine, 3-deazaguanine, and 3-deazaadenine.

Nucleotide analogs can also include modifications of the sugar moiety. Modifications to the sugar moiety include, but are not limited to, natural modifications of the ribose and deoxy ribose as well as synthetic modifications. Sugar modifications include, but are not limited to, the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl, and alkynyl may be substituted or unsubstituted C₁₋₁₀alkyl or C₂₋₁₀alkenyl, and C₂₋₁₀alkynyl. Exemplary 2′ sugar modifications also include, but are not limited to, —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m, independently, are from 1 to about 10. Other modifications at the 2′ position include, but are not limited to, C₁₋₁₀alkyl, substituted lower alkyl, alkaryl, aralkyl, O-alkaryl or O-aralkyl, SH, SCH₃, OCN, CI, Br, CN, CF₃, OCF₃, SOCH₃, SO₂CH₃, ONO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars can also include those that contain modifications at the bridging ring oxygen, such as CH₂ and S. Nucleotide sugar analogs can also have sugar mimetics, such as cyclobutyl moieties in place of the pentofuranosyl sugar.

Nucleotide analogs can also be modified at the phosphate moiety. Modified phosphate moieties include, but are not limited to, those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. These phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts, and free acid forms are also included. Nucleotide substitutes also include peptide nucleic acids (PNAs).

In some embodiments, the saRNA molecules have termini modifications. In some embodiments, the 5′ end of the antisense strand is phosphorylated. In some embodiments, 5′-phosphate analogs that cannot be hydrolyzed, such as 5′-(E)-vinyl-phosphonate are used. In some embodiments, the saRNA molecules have backbone modifications. In some embodiments, the modified phosphodiester groups that link consecutive ribose nucleosides have been shown to enhance the stability and in vivo bioavailability of siRNAs The non-ester groups (—OH, ═O) of the phosphodiester linkage can be replaced with sulfur, boron, or acetate to give phosphorothioate, boranophosphate, and phosphonoacetate linkages. In addition, substituting the phosphodiester group with a phosphotriester can facilitate cellular uptake of siRNAs and retention on serum components by eliminating their negative charge. In some embodiments, the siRNA molecules have sugar modifications. In some embodiments, the sugars are deprotonated (reaction catalyzed by exo- and endonucleases) whereby the 2′-hydroxyl can act as a nucleophile and attack the adjacent phosphorous in the phosphodiester bond. Such alternatives include 2′-O-methyl, 2′-O-methoxyethyl, and 2′-fluoro modifications.

In some embodiments, the saRNA molecules have base modifications. In some embodiments, the bases can be substituted with modified bases such as pseudouridine, 5′-methylcytidine, N6-methyladenosine, inosine, and N7-methylguanosine.

In some embodiments, the saRNA molecules are conjugated to lipids. Lipids can be conjugated to the 5′ or 3′ termini of saRNA to improve their in vivo bioavailability by allowing them to associate with serum lipoproteins. Representative lipids include, but are not limited to, cholesterol and vitamin E, and fatty acids, such as palmitate and tocopherol.

In some embodiments, a representative saRNA has the following formula:

Sense:
mN*mN*/i2FN/mN/i2FN/mN/i2FN/mN/i2FN/mN/i2FN/
mN/i2FN/mN/i2FN/mN/i2FN/*mN*/32FN/
Antisense:
/52FN/*/i2FN/*mN/i2FN/mN/i2FN/mN/i2FN/mN/
i2FN/mN/i2FN/mN/i2FN/mN/i2FN/mN/i2FN/mN*N*N

wherein: “N” is the base; “2F” is a 2′-F modification; “m” is a 2′-O-methyl modification, “I” is an internal base; and “*” is a phosphorothioate backbone linkage.

In any of the embodiments described herein, the saRNA molecules may be administered, for example, as one to two hour i.v. infusions or s.c. injections. In any of the embodiments described herein, the saRNA molecules may be administered at dose levels that range from about 50 mg to about 900 mg, from about 100 mg to about 800 mg, from about 150 mg to about 700 mg, or from about 175 to about 640 mg (2.5 to 9.14 mg/kg; 92.5 to 338 mg/m²—based on an assumption of a body weight of 70 kg and a conversion of mg/kg to mg/m² dose levels based on a mg/kg dose multiplier value of 37 for humans).

The present disclosure also provides vectors comprising any one or more of the saRNA molecules. In some embodiments, the vectors comprise any one or more of the saRNA molecules and a heterologous nucleic acid. The vectors can be viral or nonviral vectors capable of transporting a nucleic acid molecule. In some embodiments, the vector is a plasmid or cosmid (such as, for example, a circular double-stranded DNA into which additional DNA segments can be ligated). In some embodiments, the vector is a viral vector, wherein additional DNA segments can be ligated into the viral genome. Expression vectors include, but are not limited to, plasmids, cosmids, retroviruses, adenoviruses, adeno-associated viruses (AAV), plant viruses such as cauliflower mosaic virus and tobacco mosaic virus, yeast artificial chromosomes (YACs), Epstein-Barr (EBV)-derived episomes, and other expression vectors known in the art.

The present disclosure also provides compositions comprising any one or more of the saRNA molecules. In some embodiments, the composition is a pharmaceutical composition. In some embodiments, the compositions comprise a carrier and/or excipient. Examples of carriers include, but are not limited to, poly(lactic acid) (PLA) microspheres, poly(D,L-lactic-coglycolic-acid) (PLGA) microspheres, liposomes, micelles, inverse micelles, lipid cochleates, and lipid microtubules. A carrier may comprise a buffered salt solution such as PBS, HBSS, etc.

In any of the methods of treatment or prevention described herein, the subject being treated may comprise an HNF4A variant nucleic acid molecule. In some embodiments, the subject being treated is heterozygous for the HNF4A variant nucleic acid molecule. In some embodiments, the subject being treated is homozygous for the HNF4A variant nucleic acid molecule. In some embodiments, the subject being treated is HNF4A reference. The HNF4A variant nucleic acid molecule can be any of the HNF4A variant nucleic acid molecules disclosed herein. In some embodiments, the HNF4A variant nucleic acid molecule is a HNF4A variant genomic nucleic acid molecule that comprises the genetic variation rs150776703, or is an mRNA molecule produced therefrom, or is a cDNA molecule produced from the mRNA molecule.

In some embodiments, the methods of treatment or prevention further comprise detecting the presence or absence of an HNF4A variant nucleic acid molecule in a biological sample from the subject. In some embodiments, the HNF4A variant nucleic acid molecule can be any of the HNF4A variant nucleic acid molecules disclosed herein. In some embodiments, the HNF4A variant nucleic acid molecule is a HNF4A variant genomic nucleic acid molecule that comprises the genetic variation rs150776703, or is an mRNA molecule produced therefrom, or is a cDNA molecule produced from the mRNA molecule.

The present disclosure also provides methods of treating a subject with a Type 2 diabetes and/or chronic kidney disease therapeutic agent, wherein the subject has Type 2 diabetes and/or chronic kidney disease or is at risk of developing Type 2 diabetes and/or chronic kidney disease. The methods comprise determining whether the subject has an HNF4A variant nucleic acid molecule by obtaining or having obtained a biological sample from the subject, and performing or having performed a sequence analysis on the biological sample to determine if the subject has a genotype comprising the HNF4A variant nucleic acid molecule. In embodiments where the subject is HNF4A reference, the methods further comprise administering or continuing to administer the Type 2 diabetes and/or chronic kidney disease therapeutic agent in an amount that is the same as or less than a standard dosage amount to the subject, and/or administering an HNF4A agonist to the subject. In embodiments where the subject is heterozygous for the HNF4A variant nucleic acid molecule, the methods further comprise administering or continuing to administer the Type 2 diabetes and/or chronic kidney disease therapeutic agent in an amount that is the same as or less than a standard dosage amount to the subject, and/or administering an HNF4A agonist to the subject. In embodiments where the subject is homozygous for the HNF4A variant nucleic acid molecule, the methods further comprise administering or continuing to administer the Type 2 diabetes and/or chronic kidney disease therapeutic agent in a standard dosage amount to the subject. The presence of an HNF4A variant nucleic acid molecule indicates the subject has a decreased risk of developing Type 2 diabetes and/or chronic kidney disease (compared to subjects that are HNF4A reference). In some embodiments, the subject is HNF4A reference. In some embodiments, the subject is heterozygous for an HNF4A variant nucleic acid molecule. In some embodiments, the subject is homozygous for an HNF4A variant nucleic acid molecule. In any of the embodiments described herein, the HNF4A agonist is an example of a Type 2 diabetes and/or chronic kidney disease therapeutic agent. In some embodiments, the HNF4A variant nucleic acid molecule is a HNF4A variant genomic nucleic acid molecule that comprises the genetic variation rs150776703, or is an mRNA molecule produced therefrom, or is a cDNA molecule produced from the mRNA molecule.

For subjects that are genotyped or determined to be either HNF4A reference or heterozygous for an HNF4A variant nucleic acid molecule, such subjects can be administered an HNF4A agonist, as described herein.

Detecting the presence or absence of an HNF4A variant nucleic acid molecule in a biological sample from a subject and/or determining whether a subject has an HNF4A variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

In some embodiments, when the subject is HNF4A reference, the subject is administered a Type 2 diabetes and/or chronic kidney disease therapeutic agent in an amount that is the same as or less than a standard dosage amount, and/or an HNF4A agonist. In some embodiments, when the subject is heterozygous for an HNF4A variant nucleic acid molecule, the subject is administered a Type 2 diabetes therapeutic agent and/or chronic kidney disease in an amount that is the same as or less than a standard dosage amount, and/or an HNF4A agonist.

In some embodiments, the treatment or prevention methods comprise detecting the presence or absence of an increase in the expression of an HNF4A variant mRNA or polypeptide in a biological sample from the subject. In some embodiments, when the subject does not have an increase in the expression of an HNF4A variant mRNA or polypeptide, the subject is administered a Type 2 diabetes and/or chronic kidney disease therapeutic agent in an amount that is the same as or less than a standard dosage amount, and/or an HNF4A agonist. In some embodiments, when the subject has an increase in the expression of an HNF4A variant mRNA or polypeptide, the subject is administered a Type 2 diabetes and/or chronic kidney disease therapeutic agent in a standard dosage amount.

The present disclosure also provides methods of treating a subject with a Type 2 diabetes and/or chronic kidney disease therapeutic agent, wherein the subject has Type 2 diabetes and/or chronic kidney disease or is at risk of developing Type 2 diabetes and/or chronic kidney disease. The methods comprise determining whether the subject has an increase in the expression of an HNF4A variant mRNA or polypeptide by obtaining or having obtained a biological sample from the subject, and performing or having performed an assay on the biological sample to determine if the subject has an increase in the expression of an HNF4A variant mRNA or polypeptide. In embodiments where the subject does not have an increase in the expression of an HNF4A variant mRNA or polypeptide, the methods further comprise administering or continuing to administer the Type 2 diabetes and/or chronic kidney disease therapeutic agent in an amount that is the same as or less than a standard dosage amount to the subject, and/or administering an HNF4A agonist to the subject. In embodiments where the subject has an increase in the expression of an HNF4A variant mRNA or polypeptide, the methods further comprise administering or continuing to administer the Type 2 diabetes and/or chronic kidney disease therapeutic agent in a standard dosage amount to the subject. The presence of an increase in the expression of an HNF4A variant mRNA or polypeptide indicates the subject has a decreased risk of developing Type 2 diabetes and/or chronic kidney disease (compared to subjects that are HNF4A reference). In some embodiments, the subject has an increase in the expression of an HNF4A variant mRNA or polypeptide. In some embodiments, the subject does not have an increase in the expression of an HNF4A variant mRNA or polypeptide. In any of the embodiments described herein, the HNF4A agonist is an example of a Type 2 diabetes and/or chronic kidney disease therapeutic agent. In some embodiments, the HNF4A variant nucleic acid molecule is a HNF4A variant genomic nucleic acid molecule that comprises the genetic variation rs150776703, or is an mRNA molecule produced therefrom, or is a cDNA molecule produced from the mRNA molecule.

Detecting an increase in the expression of an HNF4A variant mRNA or polypeptide can be carried out by a variety of known methods. In some embodiments, these methods can be carried out in vitro. In some embodiments, these methods can be carried out in situ. In some embodiments, these methods can be carried out in vivo. In any of these embodiments, the mRNA or polypeptide can be present within a cell obtained from the subject.

In some embodiments, the treatment or prevention methods comprise detecting the presence or absence of an HNF4A variant polypeptide in a biological sample from the subject. In some embodiments, when the subject does not have an HNF4A variant polypeptide, the subject is administered a Type 2 diabetes and/or chronic kidney disease therapeutic agent in an amount that is the same as or less than a standard dosage amount, and/or an HNF4A agonist. In some embodiments, when the subject has an HNF4A variant polypeptide, the subject is administered a Type 2 diabetes and/or chronic kidney disease therapeutic agent in standard dosage amount. In some embodiments, the HNF4A variant polypeptide is HNF4A Pro437Ser.

The present disclosure also provides methods of treating a subject with a Type 2 diabetes and/or chronic kidney disease therapeutic agent, wherein the subject has Type 2 diabetes and/or chronic kidney disease or is at risk of developing Type 2 diabetes and/or chronic kidney disease. The methods comprise determining whether the subject has an HNF4A variant polypeptide by obtaining or having obtained a biological sample from the subject and performing or having performed an assay on the biological sample to determine if the subject has an HNF4A variant polypeptide. When the subject does not have an HNF4A variant polypeptide, the subject is administered the Type 2 diabetes and/or chronic kidney disease therapeutic agent in an amount that is the same as or less than a standard dosage amount, and/or an HNF4A agonist. When the subject has an HNF4A variant polypeptide, the subject is administered the Type 2 diabetes and/or chronic kidney disease therapeutic agent in a standard dosage amount. The presence of an HNF4A variant polypeptide indicates the subject has a decreased risk of developing Type 2 diabetes and/or chronic kidney disease (compared to subjects that are HNF4A reference). In some embodiments, the subject has an HNF4A variant polypeptide. In some embodiments, the subject does not have an HNF4A variant polypeptide. In some embodiments, the HNF4A variant polypeptide is HNF4A Pro437Ser.

The present disclosure also provides methods of preventing a subject from developing Type 2 diabetes and/or chronic kidney disease by administering a Type 2 diabetes and/or chronic kidney disease therapeutic agent. In some embodiments, the method comprises determining whether the subject has an HNF4A variant polypeptide by obtaining or having obtained a biological sample from the subject and performing or having performed an assay on the biological sample to determine if the subject has an HNF4A variant polypeptide. When the subject does not have an HNF4A variant polypeptide, the subject is administered the Type 2 diabetes and/or chronic kidney disease therapeutic agent in an amount that is the same as or less than a standard dosage amount, and/or an HNF4A agonist. When the subject has an HNF4A variant polypeptide, the subject is administered the Type 2 diabetes and/or chronic kidney disease therapeutic agent in a standard dosage amount. The presence of an HNF4A variant polypeptide indicates the subject has a decreased risk of developing Type 2 diabetes and/or chronic kidney disease (compared to subjects that are HNF4A reference). In some embodiments, the subject has an HNF4A variant polypeptide. In some embodiments, the subject does not have an HNF4A variant polypeptide. In some embodiments, the HNF4A variant polypeptide is HNF4A Pro437Ser.

Detecting the presence or absence of an HNF4A variant polypeptide in a biological sample from a subject and/or determining whether a subject has an HNF4A variant polypeptide can be carried out by any of the methods described herein. In some embodiments, these methods can be carried out in vitro. In some embodiments, these methods can be carried out in situ. In some embodiments, these methods can be carried out in vivo. In any of these embodiments, the polypeptide can be present within a cell obtained from the subject.

In some embodiments, the Type 2 diabetes and/or chronic kidney disease therapeutic agent is wild type HNF4A protein. In some embodiments, CRISPR technology or viral vector technology can be used to introduce the gene encoding wild type HNF4A protein into a cell, either with chromosomal insertion or extra-chromosomally, to produce wild type HNF4A protein within one or more cells of a subject.

In some embodiments, the Type 2 diabetes therapeutic agents include, but are not limited to, GLUCOPHAGE® and GLUMETZA® (metformin); a sulfonylurea such as DIABETA® and GLYNASE® (glyburide), GLUCOTROL® (glipizide) and AMARYL® (glimepiride); a meglitinide such as PRANDIN® (repaglinide) and STARLIX® (nateglinide); a thiazolidinedione such as AVANDIA® (rosiglitazone) and ACTOS® (pioglitazone); a dipeptidyl peptidase-4 (DPP-4) inhibitor such as JANUVIA® (sitagliptin), ONGLYZA® (saxagliptin) and TRADJENTA® (linagliptin); a glucagon-like peptide-1 (GLP-1) receptor agonist such as BYETTA® (exenatide) and VICTOZA® (liraglutide); an SGLT2 inhibitor such as INVOKANA® (canagliflozin) and FARXIGA® (dapagliflozin); APIDRA® (insulin glulisine), HUMALOG® (insulin lispro), NOVOLOG® (insulin aspart), LANTUS® (insulin glargine), LEVEMIR® (insulin detemir), or HUMULIN® N and NOVOLIN® N (insulin isophane), or PRALUENT® (alirocumab), or any combination thereof.

In some embodiments, the Type 2 diabetes therapeutic agents include, but are not limited to, metformin, a sulfonylurea (glyburide, glipizide, and glimepiride), a meglitinide (repaglinide and nateglinide), a thiazolidinediones (rosiglitazone and pioglitazone), a DPP-4 inhibitor (sitagliptin, saxagliptin and linagliptin), a GLP-1 receptor agonist (exenatide and liraglutide), an SGLT2 inhibitor (canagliflozin and dapagliflozin), or insulin glulisine, insulin lispro, insulin aspart, insulin glargine, insulin detemir, or insulin isophane, or alirocumab, or any combination thereof. In some embodiments, the Type 2 diabetes therapeutic agent comprises metformin. In some embodiments, the Type 2 diabetes therapeutic agent comprises a sulfonylurea (glyburide, glipizide, and glimepiride). In some embodiments, the Type 2 diabetes therapeutic agent comprises glyburide. In some embodiments, the Type 2 diabetes therapeutic agent comprises glipizide. In some embodiments, the Type 2 diabetes therapeutic agent comprises glimepiride. In some embodiments, the Type 2 diabetes therapeutic agent comprises a meglitinide (repaglinide and nateglinide). In some embodiments, the Type 2 diabetes therapeutic agent comprises repaglinide. In some embodiments, the Type 2 diabetes therapeutic agent comprises nateglinide. In some embodiments, the Type 2 diabetes therapeutic agent comprises a thiazolidinedione (rosiglitazone and pioglitazone). In some embodiments, the Type 2 diabetes therapeutic agent comprises rosiglitazone. In some embodiments, the Type 2 diabetes therapeutic agent comprises pioglitazone. In some embodiments, the Type 2 diabetes therapeutic agent comprises a DPP-4 inhibitor (sitagliptin, saxagliptin and linagliptin). In some embodiments, the Type 2 diabetes therapeutic agent comprises sitagliptin. In some embodiments, the Type 2 diabetes therapeutic agent comprises saxagliptin. In some embodiments, the Type 2 diabetes therapeutic agent comprises linagliptin. In some embodiments, the Type 2 diabetes therapeutic agent comprises a GLP-1 receptor agonist (exenatide and liraglutide). In some embodiments, the Type 2 diabetes therapeutic agent comprises exenatide. In some embodiments, the Type 2 diabetes therapeutic agent comprises liraglutide. In some embodiments, the Type 2 diabetes therapeutic agent comprises an SGLT2 inhibitor (canagliflozin and dapagliflozin). In some embodiments, the Type 2 diabetes therapeutic agent comprises canagliflozin. In some embodiments, the Type 2 diabetes therapeutic agent comprises dapagliflozin. In some embodiments, the Type 2 diabetes therapeutic agent comprises insulin glulisine. In some embodiments, the Type 2 diabetes therapeutic agent comprises insulin lispro. In some embodiments, the Type 2 diabetes therapeutic agent comprises insulin aspart. In some embodiments, the Type 2 diabetes therapeutic agent comprises insulin glargine. In some embodiments, the Type 2 diabetes therapeutic agent comprises insulin detemir. In some embodiments, the Type 2 diabetes therapeutic agent comprises insulin isophane. In some embodiments, the Type 2 diabetes therapeutic agent comprises alirocumab.

In some embodiments, the chronic kidney disease therapeutic agent comprises an ALDH1L1 inhibitor, an ALDOB inhibitor, a G6PC inhibitor, an LRP2 inhibitor, an RPL3L inhibitor, an SLC25A45 inhibitor, and/or an SLC7A9 inhibitors, or any combination thereof. In some embodiments, the chronic kidney disease therapeutic agent comprises an SGLT2 inhibitor such as, for example, canagliflozin, dapagliflozin, empagliflozin, ipragliflozin, luseogliflozin, or tofogliflozin, or any combination thereof.

Examples of therapeutic agents that treat or inhibit chronic kidney disease include, but are not limited to, erythropoietin, a diuretic (such as, for example, furosemide, bumetanide, ethacrynic acid, metolazone, and hydrochlorothiazide), a blood pressure medication, a phosphate binder, sodium bicarbonate, a cholesterol medication, and a gliflozin, or any combination thereof.

In some embodiments, the Type 2 diabetes and/or chronic kidney disease therapeutic agent can be combined with an HNF4A agonist. These treatments may be delayed or avoided altogether by treatment with an HNF4A agonist as described herein.

In some embodiments, the dose of the Type 2 diabetes and/or chronic kidney disease therapeutic agents can be decreased by about 10%, by about 20%, by about 30%, by about 40%, by about 50%, by about 60%, by about 70%, by about 80%, or by about 90% for subjects that are heterozygous for an HNF4A variant nucleic acid molecule or HNF4A reference (i.e., a less than the standard dosage amount) compared to subjects that are homozygous for an HNF4A variant nucleic acid molecule (who may receive a standard dosage amount). In some embodiments, the dose of the Type 2 diabetes and/or chronic kidney disease therapeutic agents can be decreased by about 10%, by about 20%, by about 30%, by about 40%, or by about 50%. In addition, subjects that are heterozygous for an HNF4A variant nucleic acid molecule or HNF4A reference can be administered the Type 2 diabetes and/or chronic kidney disease therapeutic agents less frequently compared to subjects that are homozygous for the HNF4A variant nucleic acid molecule.

Administration of the Type 2 diabetes and/or chronic kidney disease therapeutic agents and/or HNF4A agonists can be repeated, for example, after one day, two days, three days, five days, one week, two weeks, three weeks, one month, five weeks, six weeks, seven weeks, eight weeks, two months, or three months. The repeated administration can be at the same dose or at a different dose. The administration can be repeated once, twice, three times, four times, five times, six times, seven times, eight times, nine times, ten times, or more. For example, according to certain dosage regimens a subject can receive therapy for a prolonged period of time such as, for example, 6 months, 1 year, or more.

Administration of the Type 2 diabetes and/or chronic kidney disease therapeutic agents and/or HNF4A agonists can occur by any suitable route including, but not limited to, parenteral, intravenous, oral, subcutaneous, intra-arterial, intracranial, intrathecal, intraperitoneal, topical, intranasal, or intramuscular. Pharmaceutical compositions for administration are desirably sterile and substantially isotonic and manufactured under GMP conditions. Pharmaceutical compositions can be provided in unit dosage form (i.e., the dosage for a single administration). Pharmaceutical compositions can be formulated using one or more physiologically and pharmaceutically acceptable carriers, diluents, excipients, or auxiliaries. The formulation depends on the route of administration chosen. The term “pharmaceutically acceptable” means that the carrier, diluent, excipient, or auxiliary is compatible with the other ingredients of the formulation and not substantially deleterious to the recipient thereof.

The terms “treat”, “treating”, and “treatment” and “prevent”, “preventing”, and “prevention” as used herein, refer to eliciting the desired biological response, such as a therapeutic and prophylactic effect, respectively. In some embodiments, a therapeutic effect comprises one or more of a decrease/reduction in Type 2 diabetes and/or chronic kidney disease, a decrease/reduction in the severity of Type 2 diabetes and/or chronic kidney disease (such as, for example, a reduction or inhibition of development of Type 2 diabetes and/or chronic kidney disease), a decrease/reduction in symptoms and disease-related effects, delaying the onset of symptoms and disease-related effects, reducing the severity of symptoms of disease-related effects, reducing the number of symptoms and disease-related effects, reducing the latency of symptoms and disease-related effects, an amelioration of symptoms and disease-related effects, reducing secondary symptoms, reducing secondary infections, preventing relapse to Type 2 diabetes and/or chronic kidney disease, decreasing the number or frequency of relapse episodes, increasing latency between symptomatic episodes, increasing time to sustained progression, speeding recovery, or increasing efficacy of or decreasing resistance to alternative therapeutics, and/or an increased survival time of the affected host animal, following administration of the agent or composition comprising the agent. A prophylactic effect may comprise a complete or partial avoidance/inhibition or a delay of Type 2 diabetes and/or chronic kidney disease development/progression (such as, for example, a complete or partial avoidance/inhibition or a delay), and an increased survival time of the affected host animal, following administration of a therapeutic protocol. Treatment of Type 2 diabetes and/or chronic kidney disease encompasses the treatment of a subject already diagnosed as having any form of Type 2 diabetes and/or chronic kidney disease at any clinical stage or manifestation, the delay of the onset or evolution or aggravation or deterioration of the symptoms or signs of Type 2 diabetes and/or chronic kidney disease, and/or preventing and/or reducing the severity of Type 2 diabetes and/or chronic kidney disease.

In some embodiments, the HNF4A agonist and the Type 2 diabetes and/or chronic kidney disease therapeutic agent are disposed within a pharmaceutical composition. In some embodiments, the HNF4A agonist is disposed within a first pharmaceutical composition and the Type 2 diabetes and/or chronic kidney disease therapeutic agent is disposed within a second pharmaceutical composition. In some embodiments, the first pharmaceutical composition and the second pharmaceutical composition are administered simultaneously. In some embodiments, the first pharmaceutical composition is administered before the second pharmaceutical composition. In some embodiments, the first pharmaceutical composition is administered after the second pharmaceutical composition.

The present disclosure also provides methods of identifying a subject having a risk of developing Type 2 diabetes and/or chronic kidney disease. In some embodiments, the method comprises determining or having determined in a biological sample obtained from the subject the presence or absence of an HNF4A variant nucleic acid molecule (such as a genomic nucleic acid molecule, mRNA molecule, and/or cDNA molecule). When the subject lacks an HNF4A variant nucleic acid molecule (i.e., the subject is genotypically categorized as HNF4A reference), then the subject has an increased risk of developing Type 2 diabetes and/or chronic kidney disease (compared to subjects that are heterozygous or homozygous for an HNF4A variant nucleic acid molecule). When the subject has an HNF4A variant nucleic acid molecule (i.e., the subject is heterozygous or homozygous for an HNF4A variant nucleic acid molecule), then the subject has a decreased risk of developing Type 2 diabetes and/or chronic kidney disease (compared to subjects that are HNF4A reference). In some embodiments, the HNF4A variant nucleic acid molecule is a HNF4A variant genomic nucleic acid molecule that comprises the genetic variation rs150776703, or is an mRNA molecule produced therefrom, or is a cDNA molecule produced from the mRNA molecule.

Having a single copy of an HNF4A variant nucleic acid molecule is more protective of a subject from developing Type 2 diabetes and/or chronic kidney disease than having no copies of an HNF4A variant nucleic acid molecule. Without intending to be limited to any particular theory or mechanism of action, it is believed that a single copy of an HNF4A variant nucleic acid molecule (i.e., heterozygous for an HNF4A variant nucleic acid molecule) is protective of a subject from developing Type 2 diabetes and/or chronic kidney disease and it is also believed that having two copies of an HNF4A variant nucleic acid molecule (i.e., homozygous for an HNF4A variant nucleic acid molecule) may be more protective of a subject from developing Type 2 diabetes and/or chronic kidney disease, relative to a subject with a single copy. Thus, in some embodiments, a single copy of an HNF4A variant nucleic acid molecule may not be completely protective, but instead, may be partially or incompletely protective of a subject from developing Type 2 diabetes and/or chronic kidney disease. While not desiring to be bound by any particular theory, there may be additional factors or molecules involved in the development of Type 2 diabetes and/or chronic kidney disease that are still present in a subject having a single copy of an HNF4A variant nucleic acid molecule, thus resulting in less than complete protection from the development of Type 2 diabetes and/or chronic kidney disease.

Determining whether a subject has an HNF4A variant nucleic acid molecule in a biological sample from a subject and/or determining whether a subject has an HNF4A variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

In some embodiments, when a subject is identified as having an increased risk of developing Type 2 diabetes and/or chronic kidney disease, the subject is administered a Type 2 diabetes and/or chronic kidney disease therapeutic agent, and/or an HNF4A agonist, as described herein. For example, when the subject is HNF4A reference, and therefore has an increased risk of developing Type 2 diabetes and/or chronic kidney disease (compared to subjects that are heterozygous or homozygous for an HNF4A variant nucleic acid molecule), the subject is administered a Type 2 diabetes and/or chronic kidney disease therapeutic agent in an amount that is the same as or less than a standard dosage amount, and/or is administered an HNF4A agonist. In some embodiments, when the subject is heterozygous for an HNF4A variant nucleic acid molecule, the subject is administered the Type 2 diabetes and/or chronic kidney disease therapeutic agent in an amount that is the same as or less than a standard dosage amount, and/or is administered an HNF4A agonist. In some embodiments, when the subject is homozygous for an HNF4A variant nucleic acid molecule, the subject is administered a Type 2 diabetes and/or chronic kidney disease therapeutic agent in a standard dosage amount. In some embodiments, the subject is HNF4A reference. In some embodiments, the subject is heterozygous for an HNF4A variant nucleic acid molecule. In some embodiments, the subject is homozygous for an HNF4A variant nucleic acid molecule.

The present disclosure also provides methods of detecting the presence or absence of an HNF4A variant nucleic acid molecule (i.e., a genomic nucleic acid molecule, an mRNA molecule, or a cDNA molecule produced from an mRNA molecule) in a biological sample from a subject. It is understood that gene sequences within a population and mRNA molecules encoded by such genes can vary due to polymorphisms such as single-nucleotide polymorphisms.

The biological sample can be derived from any cell, tissue, or biological fluid from the subject. The biological sample may comprise any clinically relevant tissue, such as a bone marrow sample, a tumor biopsy, a fine needle aspirate, or a sample of bodily fluid, such as blood, gingival crevicular fluid, plasma, serum, lymph, ascitic fluid, cystic fluid, or urine. In some cases, the sample comprises a buccal swab. The biological sample used in the methods disclosed herein can vary based on the assay format, nature of the detection method, and the tissues, cells, or extracts that are used as the sample. A biological sample can be processed differently depending on the assay being employed. For example, when detecting any HNF4A variant nucleic acid molecule, preliminary processing designed to isolate or enrich the biological sample for the genomic DNA can be employed. A variety of techniques may be used for this purpose. When detecting the level of any HNF4A variant nucleic acid molecule, different techniques can be used enrich the biological sample with mRNA molecules. Various methods to detect the presence or level of an mRNA molecule or the presence of a particular variant genomic DNA locus can be used.

In some embodiments, detecting an HNF4A variant nucleic acid molecule in a subject comprises performing a sequence analysis on a biological sample obtained from the subject to determine whether an HNF4A genomic nucleic acid molecule in the biological sample, and/or an HNF4A mRNA molecule in the biological sample, and/or an HNF4A cDNA molecule produced from an mRNA molecule in the biological sample, is present in the sample. In some embodiments, the methods detect the HNF4A variant genomic nucleic acid molecule that comprises the genetic variation rs150776703, or an mRNA molecule produced therefrom, or a cDNA molecule produced from the mRNA molecule.

In some embodiments, the methods of detecting the presence or absence of an HNF4A variant nucleic acid molecule (such as, for example, a genomic nucleic acid molecule, an mRNA molecule, and/or a cDNA molecule produced from an mRNA molecule) in a subject comprise performing an assay on a biological sample obtained from the subject. The assay determines whether a nucleic acid molecule in the biological sample comprises a particular nucleotide sequence.

In some embodiments, the biological sample comprises a cell or cell lysate. Such methods can further comprise, for example, obtaining a biological sample from the subject comprising an HNF4A genomic nucleic acid molecule or mRNA molecule, and if mRNA, optionally reverse transcribing the mRNA into cDNA. Such assays can comprise, for example determining the identity of these positions of the particular HNF4A nucleic acid molecule. In some embodiments, the method is an in vitro method.

In some embodiments, the determining step, detecting step, or sequence analysis comprises sequencing at least a portion of the nucleotide sequence of the HNF4A genomic nucleic acid molecule, the HNF4A mRNA molecule, or the HNF4A cDNA molecule in the biological sample that comprises a genetic variation compared to the corresponding HNF4A reference molecule. In some embodiments, the sequenced portion comprises one or more variations that cause a gain-of-function (partial or complete) or are predicted to cause a gain-of-function (partial or complete).

In some embodiments, the assay comprises sequencing the entire nucleic acid molecule. In some embodiments, only an HNF4A genomic nucleic acid molecule is analyzed. In some embodiments, only an HNF4A mRNA is analyzed. In some embodiments, only an HNF4A cDNA obtained from the HNF4A mRNA is analyzed.

Alteration-specific polymerase chain reaction techniques can be used to detect mutations such as SNPs in a nucleic acid sequence. Alteration-specific primers can be used because the DNA polymerase will not extend when a mismatch with the template is present.

In some embodiments, the nucleic acid molecule in the sample is mRNA and the mRNA is reverse-transcribed into a cDNA prior to the amplifying step. In some embodiments, the nucleic acid molecule is present within a cell obtained from the subject.

In some embodiments, the assay comprises contacting the biological sample with a primer or probe, such as an alteration-specific primer or alteration-specific probe, that specifically hybridizes to an HNF4A variant genomic sequence, variant mRNA sequence, or variant cDNA sequence and not the corresponding HNF4A reference sequence under stringent conditions and determining whether hybridization has occurred.

In some embodiments, the determining step, detecting step, or sequence analysis comprises: a) amplifying at least a portion of the HNF4A nucleic acid molecule that encodes the HNF4A polypeptide; b) labeling the amplified nucleic acid molecule with a detectable label; c) contacting the labeled nucleic acid molecule with a support comprising an alteration-specific probe; and d) detecting the detectable label.

In some embodiments, the assay comprises RNA sequencing (RNA-Seq). In some embodiments, the assays also comprise reverse transcribing mRNA into cDNA, such as by the reverse transcriptase polymerase chain reaction (RT-PCR).

In some embodiments, the methods utilize probes and primers of sufficient nucleotide length to bind to the target nucleotide sequence and specifically detect and/or identify a polynucleotide comprising an HNF4A variant genomic nucleic acid molecule, variant mRNA molecule, or variant cDNA molecule. The hybridization conditions or reaction conditions can be determined by the operator to achieve this result. The nucleotide length may be any length that is sufficient for use in a detection method of choice, including any assay described or exemplified herein. Such probes and primers can hybridize specifically to a target nucleotide sequence under high stringency hybridization conditions. Probes and primers may have complete nucleotide sequence identity of contiguous nucleotides within the target nucleotide sequence, although probes differing from the target nucleotide sequence and that retain the ability to specifically detect and/or identify a target nucleotide sequence may be designed by conventional methods. Probes and primers can have about 80%, about 85%, about 90%, about 91%, about 92%, about 93%, about 94%, about 95%, about 96%, about 97%, about 98%, about 99%, or 100% sequence identity or complementarity with the nucleotide sequence of the target nucleic acid molecule.

Illustrative examples of nucleic acid sequencing techniques include, but are not limited to, chain terminator (Sanger) sequencing and dye terminator sequencing. Other methods involve nucleic acid hybridization methods other than sequencing, including using labeled primers or probes directed against purified DNA, amplified DNA, and fixed cell preparations (fluorescence in situ hybridization (FISH)). In some methods, a target nucleic acid molecule may be amplified prior to or simultaneous with detection. Illustrative examples of nucleic acid amplification techniques include, but are not limited to, polymerase chain reaction (PCR), ligase chain reaction (LCR), strand displacement amplification (SDA), and nucleic acid sequence based amplification (NASBA). Other methods include, but are not limited to, ligase chain reaction, strand displacement amplification, and thermophilic SDA (tSDA).

In hybridization techniques, stringent conditions can be employed such that a probe or primer will specifically hybridize to its target. In some embodiments, a polynucleotide primer or probe under stringent conditions will hybridize to its target sequence to a detectably greater degree than to other non-target sequences, such as, at least 2-fold, at least 3-fold, at least 4-fold, or more over background, including over 10-fold over background. In some embodiments, a polynucleotide primer or probe under stringent conditions will hybridize to its target nucleotide sequence to a detectably greater degree than to other nucleotide sequences by at least 2-fold. In some embodiments, a polynucleotide primer or probe under stringent conditions will hybridize to its target nucleotide sequence to a detectably greater degree than to other nucleotide sequences by at least 3-fold. In some embodiments, a polynucleotide primer or probe under stringent conditions will hybridize to its target nucleotide sequence to a detectably greater degree than to other nucleotide sequences by at least 4-fold. In some embodiments, a polynucleotide primer or probe under stringent conditions will hybridize to its target nucleotide sequence to a detectably greater degree than to other nucleotide sequences by over 10-fold over background. Stringent conditions are sequence-dependent and will be different in different circumstances.

Appropriate stringency conditions which promote DNA hybridization, for example, 6× sodium chloride/sodium citrate (SSC) at about 45° C., followed by a wash of 2×SSC at 50° C., are known or can be found in Current Protocols in Molecular Biology, John Wiley & Sons, N.Y. (1989), 6.3.1-6.3.6. Typically, stringent conditions for hybridization and detection will be those in which the salt concentration is less than about 1.5 M Na⁺ ion, typically about 0.01 to 1.0 M Na⁺ ion concentration (or other salts) at pH 7.0 to 8.3 and the temperature is at least about 30° ° C. for short probes (such as, for example, 10 to 50 nucleotides) and at least about 60° C. for longer probes (such as, for example, greater than 50 nucleotides). Stringent conditions may also be achieved with the addition of destabilizing agents such as formamide. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 200, at least about 300, at least about 400, at least about 500, at least about 600, at least about 700, at least about 800, at least about 900, at least about 1000, at least about 2000, at least about 3000, at least about 4000, or at least about 5000 nucleotides. In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, or at least about 25 nucleotides. In some embodiments, the isolated nucleic acid molecules comprise or consist of at least about 18 nucleotides. In some embodiments, the isolated nucleic acid molecules comprise or consists of at least about 15 nucleotides. In some embodiments, the isolated nucleic acid molecules consist of or comprise from about 10 to about 35, from about 10 to about 30, from about 10 to about 25, from about 12 to about 30, from about 12 to about 28, from about 12 to about 24, from about 15 to about 30, from about 15 to about 25, from about 18 to about 30, from about 18 to about 25, from about 18 to about 24, or from about 18 to about 22 nucleotides. In some embodiments, the isolated nucleic acid molecules consist of or comprise from about 18 to about 30 nucleotides. In some embodiments, the isolated nucleic acid molecules comprise or consist of at least about 15 nucleotides to at least about 35 nucleotides.

In some embodiments, such isolated nucleic acid molecules hybridize to HNF4A variant nucleic acid molecules (such as genomic nucleic acid molecules, mRNA molecules, and/or cDNA molecules) under stringent conditions. Such nucleic acid molecules can be used, for example, as probes, primers, alteration-specific probes, or alteration-specific primers as described or exemplified herein, and include, without limitation primers and probes, each of which is described in more detail elsewhere herein and can be used in any of the methods described herein.

In some embodiments, the isolated nucleic acid molecules hybridize to at least about 15 contiguous nucleotides of a nucleic acid molecule that is at least about 70%, at least about 75%, at least about 80%, at least about 85%, at least about 90%, at least about 95%, at least about 96%, at least about 97%, at least about 98%, at least about 99%, or 100% identical to HNF4A variant nucleic acid molecules. In some embodiments, the isolated nucleic acid molecules consist of or comprise from about 15 to about 100 nucleotides, or from about 15 to about 35 nucleotides. In some embodiments, the isolated nucleic acid molecules consist of or comprise from about 15 to about 100 nucleotides. In some embodiments, the isolated nucleic acid molecules consist of or comprise from about 15 to about 35 nucleotides.

In some embodiments, the alteration-specific probes and alteration-specific primers comprise DNA. In some embodiments, the alteration-specific probes and alteration-specific primers comprise RNA.

In some embodiments, the probes and primers described herein (including alteration-specific probes and alteration-specific primers) have a nucleotide sequence that specifically hybridizes to any of the nucleic acid molecules disclosed herein, or the complement thereof. In some embodiments, the probes and primers specifically hybridize to any of the nucleic acid molecules disclosed herein under stringent conditions.

In some embodiments, the primers, including alteration-specific primers, can be used in second generation sequencing or high throughput sequencing. In some instances, the primers, including alteration-specific primers, can be modified. In particular, the primers can comprise various modifications that are used at different steps of, for example, Massive Parallel Signature Sequencing (MPSS), Polony sequencing, and 454 Pyrosequencing. Modified primers can be used at several steps of the process, including biotinylated primers in the cloning step and fluorescently labeled primers used at the bead loading step and detection step. Polony sequencing is generally performed using a paired-end tags library wherein each molecule of DNA template is about 135 bp in length. Biotinylated primers are used at the bead loading step and emulsion PCR. Fluorescently labeled degenerate nonamer oligonucleotides are used at the detection step. An adaptor can contain a 5′-biotin tag for immobilization of the DNA library onto streptavidin-coated beads.

The probes and primers described herein can be used to detect a nucleotide variation within any of the HNF4A variant nucleic acid molecules disclosed herein. The primers described herein can be used to amplify any HNF4A variant nucleic acid molecule, or a fragment thereof.

In the context of the disclosure “specifically hybridizes” means that the probe or primer (such as, for example, the alteration-specific probe or alteration-specific primer) does not hybridize to a nucleic acid sequence encoding an HNF4A reference genomic nucleic acid molecule, an HNF4A reference mRNA molecule, and/or an HNF4A reference cDNA molecule.

In some embodiments, the probes (such as, for example, an alteration-specific probe) comprise a label. In some embodiments, the label is a fluorescent label, a radiolabel, or biotin.

The present disclosure also provides supports comprising a substrate to which any one or more of the probes disclosed herein is attached. Solid supports are solid-state substrates or supports with which molecules, such as any of the probes disclosed herein, can be associated. A form of solid support is an array. Another form of solid support is an array detector. An array detector is a solid support to which multiple different probes have been coupled in an array, grid, or other organized pattern. A form for a solid-state substrate is a microtiter dish, such as a standard 96-well type. In some embodiments, a multiwell glass slide can be employed that normally contains one array per well.

The genomic nucleic acid molecules, mRNA molecules, and cDNA molecules can be from any organism. For example, the genomic nucleic acid molecules, mRNA molecules, and cDNA molecules can be human or an ortholog from another organism, such as a non-human mammal, a rodent, a mouse, or a rat. It is understood that gene sequences within a population can vary due to polymorphisms such as single-nucleotide polymorphisms.

Also provided herein are functional polynucleotides that can interact with the disclosed nucleic acid molecules. Examples of functional polynucleotides include, but are not limited to, antisense molecules, aptamers, ribozymes, triplex forming molecules, and external guide sequences. The functional polynucleotides can act as effectors, inhibitors, modulators, and stimulators of a specific activity possessed by a target molecule, or the functional polynucleotides can possess a de novo activity independent of any other molecules.

Percent identity (or percent complementarity) between particular stretches of nucleotide sequences within nucleic acid molecules or amino acid sequences within polypeptides can be determined routinely using BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). Herein, if reference is made to percent sequence identity, the higher percentages of sequence identity are preferred over the lower ones.

The present disclosure also provides methods of increasing the protein level of HNF4α in a subject in need thereof. The methods comprise administering an HNF4α protein encoded by an HNF4A gain-of-function variant nucleic acid molecule to the subject. In some embodiments, an HNF4α protein can be administered to the subject. In some embodiments, a nucleic acid molecule encoding an HNF4α protein can be administered to the subject. In some embodiments, the nucleic acid molecule encoding an HNF4α protein can be a vector, such as a plasmid or viral particle. In some embodiments, the subject in need thereof has Type 2 diabetes and/or chronic kidney disease or is at risk of developing Type 2 diabetes and/or chronic kidney disease. In some embodiments, the HNF4A gain-of-function variant nucleic acid molecule that encodes the HNF4α protein comprises a splice-site variant, a stop-gain variant, a start-loss variant, a stop-loss variant, a frameshift variant, a missense variant, and/or an in-frame indel variant. In some embodiments, the HNF4A gain-of-function variant nucleic acid molecule comprises the genetic variation rs150776703. Such methods can result in increased protein level of HNF4α in the subject compared to a subject administered wild-type HNF4α protein, or endogenous HNF4α protein expression in such a subject.

The present disclosure also provides Type 2 diabetes and/or chronic kidney disease therapeutic agents for use in the treatment or prevention of Type 2 diabetes and/or chronic kidney disease in a subject having an HNF4A variant nucleic acid molecule. Any of the Type 2 diabetes and/or chronic kidney disease therapeutic agents described herein can be used herein. Any of the HNF4A variant nucleic acid molecules disclosed herein can be used herein. In some embodiments, the HNF4A variant nucleic acid molecule is a HNF4A variant genomic nucleic acid molecule that comprises the genetic variation rs150776703, or is an mRNA molecule produced therefrom, or is a cDNA molecule produced from the mRNA molecule.

The present disclosure also provides uses of Type 2 diabetes and/or chronic kidney disease therapeutic agents for use in the preparation of a medicament for treating or preventing Type 2 diabetes and/or chronic kidney disease in a subject having an HNF4A variant nucleic acid molecule. Any of the Type 2 diabetes and/or chronic kidney disease therapeutic agents described herein can be used herein. Any of the HNF4A variant nucleic acid molecules disclosed herein can be used herein. In some embodiments, the HNF4A variant nucleic acid molecule is a HNF4A variant genomic nucleic acid molecule that comprises the genetic variation rs150776703, or is an mRNA molecule produced therefrom, or is a cDNA molecule produced from the mRNA molecule.

The present disclosure also provides HNF4A agonists for use in the treatment or prevention of Type 2 diabetes and/or chronic kidney disease in a subject that is HNF4A reference or is heterozygous for an HNF4A variant nucleic acid molecule. Any of the HNF4A agonists described herein can be used herein. Any of the HNF4A variant nucleic acid molecules disclosed herein can be used herein. In some embodiments, the HNF4A variant nucleic acid molecule is a HNF4A variant genomic nucleic acid molecule that comprises the genetic variation rs150776703, or is an mRNA molecule produced therefrom, or is a cDNA molecule produced from the mRNA molecule.

The present disclosure also provides HNF4A agonists in the preparation of a medicament for treating or preventing Type 2 diabetes and/or chronic kidney disease in a subject that is HNF4A reference or is heterozygous for an HNF4A variant nucleic acid molecule. Any of the HNF4A agonists described herein can be used herein. Any of the HNF4A variant nucleic acid molecules disclosed herein can be used herein. In some embodiments, the HNF4A variant nucleic acid molecule is a HNF4A variant genomic nucleic acid molecule that comprises the genetic variation rs150776703, or is an mRNA molecule produced therefrom, or is a cDNA molecule produced from the mRNA molecule.

In some embodiments, the HNF4A agonist and the Type 2 diabetes and/or chronic kidney disease therapeutic agent are disposed within a pharmaceutical composition. In some embodiments, the HNF4A agonist is disposed within a first pharmaceutical composition and the Type 2 diabetes and/or chronic kidney disease therapeutic agent is disposed within a second pharmaceutical composition. In some embodiments, the first pharmaceutical composition and the second pharmaceutical composition are administered simultaneously. In some embodiments, the first pharmaceutical composition is administered before the second pharmaceutical composition. In some embodiments, the first pharmaceutical composition is administered after the second pharmaceutical composition.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in ° C. or is at ambient temperature, and pressure is at or near atmospheric.

### EXAMPLES

### Example 1: General Methods

A south Asian ancestry cohort (known as the “BELIEVE Study”) that consisted of 51,289 samples were sequenced on an Illumina NovaSeq 6000 system on S4 flow cells. A total of 9,084 cases of Type 2 diabetes were present of which 56.6% were females. In 42,205 controls, 58.0% were females. The REGENIE software was used to perform a genome-wide logistic regression between Type 2 diabetes cases and controls separately for exome and TopMed imputed genetics data. The analysis was adjusted for age, age square, age x sex, sex, smoking and genetic principal components (10 from common variants, 20 from rare variants).

### Cell Culture

Huh7 cells were from Creative Bioarray (Catalog number: CSC-C9441L) and cultured in DMEM (Gibco, 11995) supplemented with 10% FBS, 100 U/ml penicillin and 0.1 mg/ml streptomycin.

### Plasmid Constructs

Wild-type plasmid (SEQ ID NO:1) and mutant HNF4α plasmid (SEQ ID NO:2) were generated and verified by sequencing.

### Transfection of Plasmids

Huh7 cells were transiently transfected with plasmid DNA by Mirus TransIT reagents (Mirus Bio). Forty-eight hours after transfection, cells were collected and an aliquot taken for RNA extraction, the rest of the cells were used for protein extraction including fractionation for cytosolic and nuclear extracts.

### Cell Fractionation & Lysate Preparation

Cells transiently transfected with HNF4α plasmids were harvested in ice cold 1×PBS and split into 2 fractions. One third were recovered in 1×RIPA buffer (BioRad)+protease inhibitors (Roche) for whole cell lysates. The other two-thirds were processed using the NE-PER nuclear and cytoplasmic extraction reagents (ThermoFisher) as per manufacturer's instructions. Briefly, cells were resuspended in CI buffer+protease inhibitors (Roche) and left to solubilize. CII buffer was then added and cells were incubated on ice for 1 minute, followed by centrifugation at full speed for 10 minutes at 4 C. The supernatant containing cytosolic extract was transferred to a new Eppendorf tube. The pellet was resuspended in NEB buffer, and left to incubate on ice for 40 minutes with vortexing every 10 minutes. The nuclear lysates were centrifuged at full speed for 10 minutes at 4 C, and the supernatant containing nuclear protein was transferred to a new Eppendorf tube. All samples were then prepared for western blotting.

### Western Blotting

Protein samples (whole cell, nuclear or cytosolic) were assessed by BCA assay (Pierce Scientific) for protein concentration and lysates were made by addition of 6× laemmli buffer (Thermo Fisher). Samples were heated at 70 C for 10 minutes, resolved by SDS-PAGE and transferred to PVDF membranes. Immunoblotting was performed with primary antibodies against HNF4α (1:1000 dilution, AB41898, Abcam), SP1 (1:1000 dilution, 9389S, Cell Signaling Technologies) and β-actin-conjugated to HRP (1:1000 dilution, 5125S, Cell Signaling Technologies). Secondary anti-rabbit or anti-mouse antibodies conjugated to HRP (1:5000 dilution; PI-1000, PI-2000, Vector Laboratories) were used and signals detected using chemiluminescent detection.

### RNA Extraction

Huh7 cells transiently transfected with HNF4α were also processed for RNA extraction. Cell pellets were lysed in QIAzol lysis reagent (Qiagen) and RNA was extracted using the RNeasy plus universal kit (Qiagen) as per manufacturer's instructions. Briefly, after lysing, cells were incubated at room temperature and genomic DNA was removed by addition of gDNA eliminator solution and incubation at room temperature. Chloroform was then added, and samples were mixed and centrifuged at 12,000×g at 4 C. The upper layer was then transferred to new Eppendorf tubes and mixed with 1:1 volume 70% ethanol. The solution was then transferred to collection tubes and centrifuged to collect RNA on the filter of a spin column. The RNA was then washed with RW1 buffer, followed by 2 washed with RPE buffer. Finally, RNA was eluted in nuclease free water and concentration was analyzed by nanodrop.

cDNA Preparation and qPCR

500 ng of RNA was reverse transcribed using the high-capacity cDNA reverse transcription kit (4374966, Thermo Fisher) to generate cDNA for qPCR. Quantitative PCR was performed in three technical replicates per sample using SYBR green (Thermo Fisher) using the Applied BiosystemsQuantStudio 7 (Thermo Fisher). The following primers were used:

1)
HNF4α:
(SEQ ID NO: 3)
5′-GAAGATTGCCAGCATCGCAG-3′
and
(SEQ ID NO: 4)
5′-GTACTTGGCCCACTCAACGA-3′;
2)
PPIA:
(SEQ ID NO: 5)
5′-GGCAAATGCTGGACCCAACAC-3′
and
(SEQ ID NO: 6)
5′-TGCTGGTCTTGCCATTCCTGG-3′

Target gene expression was assessed using the ΔΔCₜ method. The expression of HNF4α was normalized to the housekeeping gene PPIA.

### Example 2: Association of Gain-of-Function Variant with Protection Against Type 2 Diabetes

A rare missense variant 20:44429549:C:T (p.Pro437Ser) in HNF4A gene showed protective association with Type 2 diabetes (p value=8.6e-14, odd ratio (OR)=0.47, confidence intervals=0.38 to 0.57) (see, Table 1). To our knowledge, it is the first ever report of naturally occurring rare missense protective variant for Type 2 diabetes. This variant also showed a protective association with chronic kidney disease (p value=0.00079, effect=0.48, confidence intervals=0.31 to 0.73), upon adjustment for diabetes the variant remain significantly association with chronic kidney disease (p value=0.0098, effect=0.551, confidence intervals=0.35 to 0.86 (see, Table 1).

The allele frequency of variant 20:44429549:C:T is 0.01 in the Bangladesh population compared to other south Asian ancestries (0.005187) and Non-finish Europeans (0.000014), the latter two estimates are gathered from gnomAD public genomic data base (see, world wide web at “gnomad.broadinstitute.org/variant/20-44429549-C-T?dataset=gnomad_r3”).

TABLE 1
Association of HNF4A missense variant with Type 2 diabetes and chronic
kidney disease in south Asian population cohort (BELIEVE Cohort,
Genetic variant = 20:44429549:C:T; Gene = HNF4A)

| | | | | | | |
|---|---|---|---|---|---|---|
| Cases | Controls | OR | LCI | UCI | P value | AAF |

Type 2 diabetes association results

| | | | | | | |
|---|---|---|---|---|---|---|
| 8972 \| 112 \| 0 | 44070 \| 1121 \| 14 | 0.473 | 0.38 | 0.57 | 8.62059e−14 | 0.01 |

Chronic kidney disease association results

| | | | | | | |
|---|---|---|---|---|---|---|
| 1633 \| 18 \| 0 | 51409 \| 1215 \| 14 | 0.477 | 0.31 | 0.73 | 0.00079 | 0.01 |

Chronic kidney disease association results adjusted for Type 2 diabetes

| | | | | | | |
|---|---|---|---|---|---|---|
| 1633 \| 18 \| 0 | 51409 \| 1215 \| 14 | 0.551 | 0.35 | 0.86 | 0.0098 | 0.01 |

OR: odd ratio column represents the effect of alternate allele (T) on protection to Type 2 diabetes and/or chronic kidney disease when compared with controls and controlling for covariates in logistic regression test. LCI: lower confidence interval. UCI: upper confidence interval. P values: from logistic regression test represent an association of statistical significance between cases (Type 2 diabetes and/or chronic kidney disease) and controls. Cases: represent counts of Type 2 diabetes cases or chronic kidney disease cases for variant allele as reference reference|reference alternate|alternate alternate. Controls: represent counts of controls for variant allele as reference reference|reference alternate|alternate alternate. AAF: Alternate allele frequency of variant 20:44429549:C:T in the Believe cohort.

### Example 3: Transient Transfection of Wild-Type (WT) or Mutant HNF4α

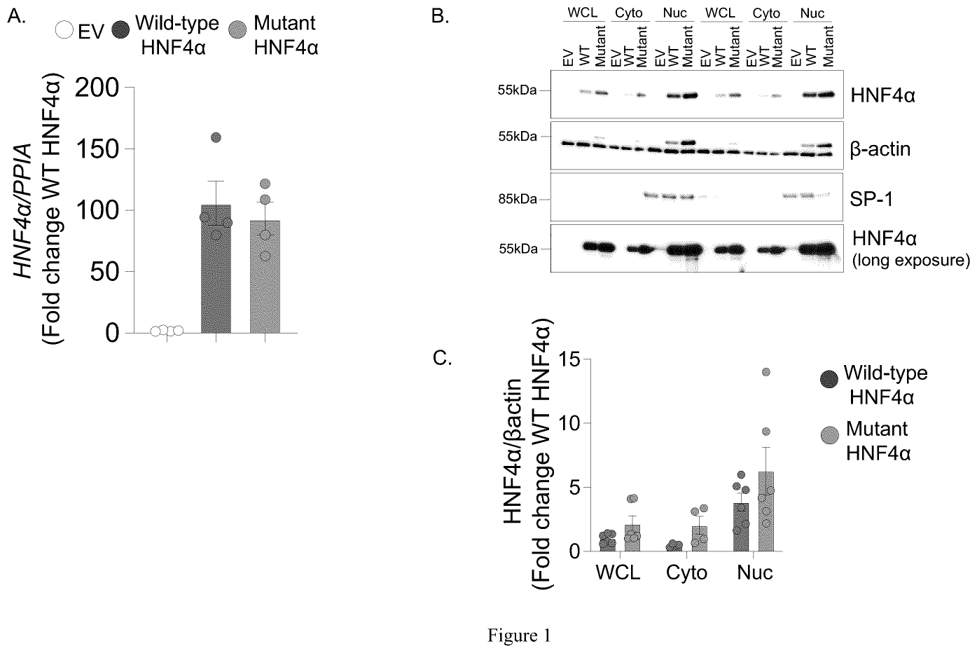

Transient transfection of either wild-type (WT) or mutant HNF4α resulted in increased HNF4α expression over endogenous levels at the RNA (see, FIG. 1, Panel A) and protein level (see, FIG. 1, Panel B), with no obvious differences in transcript expression between WT or mutant HNF4α. However, it appeared that mutant HNF4 was expressed at the protein level at a greater extent compared to that of WT HNF4α. This increased protein level was more evident in nuclear fractions as compared to whole cell or cytosolic fraction (see, FIG. 1, Panel B and Panel C). These results indicate that the mutant HNF4α construct is likely to be a gain of function mutation, due to increased stability, reduced degradation, or other post-translational modification.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety and for all purposes.

1. A method of treating a subject having Type 2 diabetes and/or chronic kidney disease or at risk of developing Type 2 diabetes and/or chronic kidney disease, the method comprising administering a Hepatocyte Nuclear Factor 4 Alpha (HNF4A) agonist to the subject.

2. The method of claim 1, wherein the HNF4A agonist comprises alverine, N-trans caffeoyltyramine (NCT), or a small activating ribonucleic acid (saRNA).

3. The method of claim 1, wherein the subject is also administered a Type 2 diabetes and/or chronic kidney disease therapeutic agent.

4. The method of claim 1, further comprising detecting the presence or absence of an HNF4A variant nucleic acid molecule in a biological sample from the subject.

5. The method of claim 4, further comprising administering a Type 2 diabetes and/or chronic kidney disease therapeutic agent in an amount that is the same as or less than a standard dosage amount to the subject when the subject is HNF4A reference or when the subject is heterozygous for the HNF4A variant nucleic acid molecule.

6. The method of claim 4, wherein the HNF4A variant nucleic acid molecule comprises a splice-site variant, a stop-gain variant, a start-loss variant, a stop-loss variant, a frameshift variant, a missense variant, and/or an in-frame indel variant.

7. The method of claim 4, wherein the HNF4A variant nucleic acid molecule comprises the genetic variation rs150776703.

8. A method of treating a subject having Type 2 diabetes and/or chronic kidney disease or at risk of developing Type 2 diabetes and/or chronic kidney disease by administering a Type 2 diabetes and/or chronic kidney disease therapeutic agent, the method comprising:
determining or having determined whether the subject has a Hepatocyte Nuclear Factor 4 Alpha (HNF4A) variant nucleic acid molecule, by:
obtaining or having obtained a biological sample from the subject; and
performing or having performed a sequence analysis on the biological sample to determine if the subject has a genotype comprising the HNF4A variant nucleic acid molecule; and
administering or continuing to administer the Type 2 diabetes and/or chronic kidney disease therapeutic agent in an amount that is the same as or less than a standard dosage amount, and/or an HNF4A agonist to a subject that is HNF4A reference or heterozygous for the HNF4A variant nucleic acid molecule; or
administering or continuing to administer the Type 2 diabetes and/or chronic kidney disease therapeutic agent in a standard dosage amount to a subject that is homozygous for the HNF4A variant nucleic acid molecule;
wherein the presence of the HNF4A variant nucleic acid molecule indicates the subject has a decreased risk of developing Type 2 diabetes and/or chronic kidney disease.

9. The method of claim 8, wherein the HNF4A agonist comprises alverine, N-trans caffeoyltyramine (NCT), or a small activating ribonucleic acid (saRNA).

10. The method of claim 8, wherein the subject is heterozygous for the HNF4A variant nucleic acid molecule, and the subject is administered or continued to be administered the Type 2 diabetes and/or chronic kidney disease therapeutic agent in an amount that is the same as or less than a standard dosage amount and the HNF4A agonist.

11. The method of claim 8, wherein the subject is HNF4A reference, and the subject is administered or continued to be administered the Type 2 diabetes and/or chronic kidney disease therapeutic agent in an amount that is the same as or less than a standard dosage amount and the HNF4A agonist.

12. The method of claim 8, wherein the HNF4A variant nucleic acid molecule comprises a splice-site variant, a stop-gain variant, a start-loss variant, a stop-loss variant, a frameshift variant, a missense variant, and/or an in-frame indel variant.

13. The method of claim 8, wherein the HNF4A variant nucleic acid molecule comprises the genetic variation rs150776703.

14-27. (canceled)

28. A method of increasing the protein level of Hepatocyte Nuclear Factor 4 Alpha (HNF4α) in a subject in need thereof, the method comprising administering an HNF4α protein encoded by an HNF4A gain-of-function variant nucleic acid molecule to the subject.

29. The method of claim 28, wherein the subject in need thereof has Type 2 diabetes and/or chronic kidney disease or is at risk of developing Type 2 diabetes and/or chronic kidney disease.

30. The method of claim 28, wherein the HNF4A gain-of-function variant nucleic acid molecule comprises a splice-site variant, a stop-gain variant, a start-loss variant, a stop-loss variant, a frameshift variant, a missense variant, and/or an in-frame indel variant.

31. The method of claim 30, wherein the HNF4A gain-of-function variant nucleic acid molecule comprises the genetic variation rs150776703.

32. The method of claim 1, wherein the subject is HNF4A reference or when the subject is heterozygous for an HNF4A variant nucleic acid molecule.

33. The method of claim 6, wherein the HNF4A variant nucleic acid molecule comprises a missense variant.

34. The method of claim 12, wherein the HNF4A variant nucleic acid molecule comprises a missense variant.

35. The method of claim 30, wherein the HNF4A variant nucleic acid molecule comprises a missense variant.


SEQUENCE LISTING (6 sequences)

SEQ ID NO: 1 - DNA, 7532 bp, synthetic construct
  source 1..7532: mol_type = other DNA
agcttaccaccatgcgactctccaaaaccctcgtcgacatggacatggccgactacagtgctgcactggacccagcctacaccaccctggaatttgagaatgtgcaggtgttgacgatgggcaatgacacgtccccatcagaaggcaccaacctcaacgcgcccaacagcctgggtgtcagcgccctgtgtgccatctgcggggaccgggccacgggcaaacactacggtgcctcgagctgtgacggctgcaagggcttcttccggaggagcgtgcggaagaaccacatgtactcctgcagatttagccggcagtgcgtggtggacaaagacaagaggaaccagtgccgctactgcaggctcaagaaatgcttccgggctggcatgaagaaggaagccgtccagaatgagcgggaccggatcagcactcgaaggtcaagctatgaggacagcagcctgccctccatcaatgcgctcctgcaggcggaggtcctgtcccgacagatcacctcccccgtctccgggatcaacggcgacattcgggcgaagaagattgccagcatcgcagatgtgtgtgagtccatgaaggagcagctgctggttctcgttgagtgggccaagtacatcccagctttctgcgagctccccctggacgaccaggtggccctgctcagagcccatgctggcgagcacctgctgctcggagccaccaagagatccatggtgttcaaggacgtgctgctcctaggcaatgactacattgtccctcggcactgcccggagctggcggagatgagccgggtgtccatacgcatccttgacgagctggtgctgcccttccaggagctgcagatcgatgacaatgagtatgcctacctcaaagccatcatcttctttgacccagatgccaaggggctgagcgatccagggaagatcaagcggctgcgttcccaggtgcaggtgagcttggaggactacatcaacgaccgccagtatgactcgcgtggccgctttggagagctgctgctgctgctgcccaccttgcagagcatcacctggcagatgatcgagcagatccagttcatcaagctcttcggcatggccaagattgacaacctgttgcaggagatgctgctgggagggtcccccagcgatgcaccccatgcccaccaccccctgcaccctcacctgatgcaggaacatatgggaaccaacgtcatcgttgccaacacaatgcccactcacctcagcaacggacagatgtccacccctgagaccccacagccctcaccgccaggtggctcagggtctgagccctataagctcctgccgggagccgtcgccacaatcgtcaagcccctctctgccatcccccagccgaccatcaccaagcaggaagttatctaggcggccgctaatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctaccggtagggcccctctcttcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgggcgcgcctcatactcctgcaggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcaggtaccaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccggatccaccatggatagatccggaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagttcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagacgcgtgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatggatctatagatctctcgtgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagtcgcgaggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgtcgaccgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctctttcctacagctcctgggcaacgtgctggttgttgtgctgtctcatcattttggcaaagaatt

SEQ ID NO: 2 - DNA, 7533 bp, synthetic construct
  source 1..7533: mol_type = other DNA
aagcttaccaccatgcgactctccaaaaccctcgtcgacatggacatggccgactacagtgctgcactggacccagcctacaccaccctggaatttgagaatgtgcaggtgttgacgatgggcaatgacacgtccccatcagaaggcaccaacctcaacgcgcccaacagcctgggtgtcagcgccctgtgtgccatctgcggggaccgggccacgggcaaacactacggtgcctcgagctgtgacggctgcaagggcttcttccggaggagcgtgcggaagaaccacatgtactcctgcagatttagccggcagtgcgtggtggacaaagacaagaggaaccagtgccgctactgcaggctcaagaaatgcttccgggctggcatgaagaaggaagccgtccagaatgagcgggaccggatcagcactcgaaggtcaagctatgaggacagcagcctgccctccatcaatgcgctcctgcaggcggaggtcctgtcccgacagatcacctcccccgtctccgggatcaacggcgacattcgggcgaagaagattgccagcatcgcagatgtgtgtgagtccatgaaggagcagctgctggttctcgttgagtgggccaagtacatcccagctttctgcgagctccccctggacgaccaggtggccctgctcagagcccatgctggcgagcacctgctgctcggagccaccaagagatccatggtgttcaaggacgtgctgctcctaggcaatgactacattgtccctcggcactgcccggagctggcggagatgagccgggtgtccatacgcatccttgacgagctggtgctgcccttccaggagctgcagatcgatgacaatgagtatgcctacctcaaagccatcatcttctttgacccagatgccaaggggctgagcgatccagggaagatcaagcggctgcgttcccaggtgcaggtgagcttggaggactacatcaacgaccgccagtatgactcgcgtggccgctttggagagctgctgctgctgctgcccaccttgcagagcatcacctggcagatgatcgagcagatccagttcatcaagctcttcggcatggccaagattgacaacctgttgcaggagatgctgctgggagggtcccccagcgatgcaccccatgcccaccaccccctgcaccctcacctgatgcaggaacatatgggaaccaacgtcatcgttgccaacacaatgcccactcacctcagcaacggacagatgtccacccctgagaccccacagccctcatcgccaggtggctcagggtctgagccctataagctcctgccgggagccgtcgccacaatcgtcaagcccctctctgccatcccccagccgaccatcaccaagcaggaagttatctaggcggccgctaatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctaccggtagggcccctctcttcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgggcgcgcctcatactcctgcaggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcaggtaccaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccggatccaccatggatagatccggaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagttcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagacgcgtgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatggatctatagatctctcgtgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagtcgcgaggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgtcgaccgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctctttcctacagctcctgggcaacgtgctggttgttgtgctgtctcatcattttggcaaagaatt

SEQ ID NO: 3 - DNA, 20 bp, synthetic construct
  source 1..20: mol_type = other DNA
gaagattgccagcatcgcag

SEQ ID NO: 4 - DNA, 20 bp, synthetic construct
  source 1..20: mol_type = other DNA
gtacttggcccactcaacga

SEQ ID NO: 5 - DNA, 21 bp, synthetic construct
  source 1..21: mol_type = other DNA
ggcaaatgctggacccaacac

SEQ ID NO: 6 - DNA, 21 bp, synthetic construct
  source 1..21: mol_type = other DNA
tgctggtcttgccattcctgg